(19) United States
(12) Patent Application Publication
(10) Pub. No.: US 2019/0198139 A1
(43) Pub. Date: Jun. 27, 2019

(54) SYSTEMS AND METHODS FOR SECURING ELECTRONIC DATA THAT INCLUDES PERSONALLY IDENTIFYING INFORMATION

(71) Applicant: CERNER INNOVATION, INC., Kansas City, KS (US)

(72) Inventors: Shreekant Majge, Malvern, PA (US); Katherine Ann Smith, Phoenixville, PA (US); Maureen Lehr, Phoenixville, PA (US)

(21) Appl. No.: 15/855,224

(22) Filed: Dec. 27, 2017

(51) Int. Cl. G16H 10/60; G16H 15/00; G06F 21/62

(52) U.S. Cl. CPC G16H 10/60; G06F 21/6254; G16H 15/00

(57) ABSTRACT

Methods, systems, and computer-readable media are disclosed herein for securing electronic data that includes personally identifying information. In embodiments, a message is obtained that includes personally identifying information encoded as electronic data. In the message, segments, fields, field components, and/or field subcomponents is/are identified that contain personally identifying information data. In embodiments, the values format(s) of those elements are recognized. The personally identifying information data is removed from the message. Then, for each of the fields, field components, and/or field subcomponents from which the personally identifying information data is removed from message, non-PHI data that conforms to the value format(s) is inserted into the message.

### BACKGROUND

Computer systems utilize, as input for executing data workflows, electronic messages that encode personally identifying information. Electronic messages encoding personally identifying information data are desirable input or required input for those computer systems. However, the personally identifying information data has a highly likelihood of being improperly breached or otherwise disclosed to a third party during execution of a data workflow. Protecting the security of that information and preventing privacy breaches of that information encoded as electronic messages is governmentally regulated and technologically challenging.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims as supported by the Specification, including the Detailed Description and Drawings.

In brief and at a high level, this disclosure describes, among other things, methods, systems, and computer-readable media for securing electronic data that includes personally identifying information. As will be described, the present invention permanently removes personally identifying information encoded in fields, field components, and/or subcomponents of a message so that the personally identifying information is unrecoverable. The claimed embodiments also generates and inserts non-personally identifying information into the message to replace the removed personally identifying information, while maintaining and conforming to the original value formats of the removed personally identifying information. As such, the invention provides a new technological function that is not found in any prior computerized systems.

A computerized method is provided in an embodiment of the present invention. The computerized method comprises obtaining a message encoding personally identifying information as data. In embodiments, the method comprises identifying personally identifying information data in the message and removing the personally identifying information data from the message. The method further comprises inserting non-personally identifying information into the message to replace the removed personally identifying information data, in embodiments.

Another embodiment provides one or more non-transitory computer-readable media having computer-executable instructions embodied thereon that, when executed, perform a method. The method comprises obtaining a Health Level Seven (HL7) message including Personal Health Information (PHI) data, in embodiments. The method identifies a field having PHI data in the HL7 message and recognizes a value format of the PHI data in the field. The method comprises removing the PHI data from the HL7 message, in embodiments. A new HL7 message is created by inserting non-PHI that conforms to the value format of the field into the HL7 message from which the PHI data is removed.

Yet another embodiment provides one or more non-transitory computer-readable media having computer-executable instructions embodied thereon that, when executed, perform a method. In accordance with the media, the method performed comprises obtaining messages that include Personal Health Information (PHI) encoded as electronic data. For each of the messages, the method comprises identifying one or more fields containing PHI data, in embodiments. For each of the one or more fields identified in each message, the method recognizes a value format of the PHI data in the field. The method removes the PHI data from each of the one or more fields identified as containing PHI data, in embodiments. For each of the one or more fields from which PHI data is removed, the method comprises inserting non-PHI data that conforms to the value format of that field into the message.

### BRIEF DESCRIPTION OF THE DRAWINGS

Embodiments are described in detail below with reference to the attached drawings figures, wherein:

FIG. 1 is a flow diagram of an exemplary method in accordance with an embodiment of the present invention;

FIG. 2 is a flow diagram of an exemplary method in accordance with an embodiment of the present invention;

FIG. 3 is a flow diagram of an exemplary method in accordance with an embodiment of the present invention;

FIG. 4 depicts an exemplary graphical user interface (GUI) in accordance with embodiments of the present invention;

FIG. 5 depicts an exemplary GUI in accordance with embodiments of the present invention;

FIG. 6 depicts an exemplary GUI in accordance with embodiments of the present invention;

FIG. 7 depicts an exemplary GUI in accordance with embodiments of the present invention;

FIG. 8 depicts an exemplary GUI in accordance with embodiments of the present invention;

FIG. 9 depicts an exemplary GUI in accordance with embodiments of the present invention;

FIG. 10 depicts an exemplary GUI in accordance with embodiments of the present invention;

FIG. 11 depicts an exemplary GUI in accordance with the embodiments of the present invention;

FIG. 12 depicts an exemplary GUI in accordance with embodiments of the present invention;

FIG. 13 depicts an exemplary GUI in accordance with embodiments of the present invention;

FIG. 14 depicts an exemplary GUI in accordance with embodiments of the present invention;

FIG. 15 depicts an exemplary GUI in accordance with embodiments of the present invention;

FIG. 16 depicts an exemplary GUI in accordance with embodiments of the present invention;

FIG. 17 depicts an exemplary GUI in accordance with embodiments of the present invention; and

FIG. 18 depicts a block diagram of an exemplary environment suitable to implement embodiments of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The present invention secures electronic data that includes personally identifying information. Electronic data that encodes personally identifying information may be compromised in computer systems that use such data as input for workflow execution. This is relevant when personally identifying information is encoded using, for example, a programming language or format that is human-readable in addition to being computer-readable. In embodiments of the invention, personally identifying information is recognized within the computer-readable language of an electronic data message (e.g., personally identifying information data can be distinguished from non-personally identifying information data), the personally identifying information data is removed from electronic data messages, and “dummy” data is intelligently added to the electronic data message as a substitute. The dummy data that is introduced into an electronic data message is relevant to the type and kind of personally identifying information data that is removed, and is further compatible with the value format that was used to encode the personally identifying information in the electronic data message. The claimed embodiments, therefore, provide new technological function(s) that is/are missing from prior computerized systems. In addition to creating new technological function(s) that ensure(s) personally identifying information data encoded in electronic data messages is not compromised, the claimed embodiments address technological privacy problems that arise from encoding personally identifying information in electronic data messages.

It should be noted that “personally identifying information”, “Personal Health Information”, “Protected Health Information”, and “PHI” are used interchangeably herein. In determining the scope of the invention, which is defined by the claims, the term “PHI” is not limited to legal, agency, and/or governmental definitions.

Personally identifying information is information that could be used, either alone or in combination with other information, to uniquely identify a person. In addition to visually or physically identifying a person based on such information, for example, personally identifying information includes information from which a person or computer may be able ascertain a person's identity based on electronically stored information. Personal Health Information is one example of personally identifying information. Examples of PHI include: a first name; a last name; age; gender; ethnicity; birthdate; social security number; medical record number; patient number; a room number; demographic information; mailing address; city and state of residence; telephone number; place of work; profession; physical descriptors such as height and weight; next of kin; familial relationships to another person; diagnosis and/or conditions; risk factors and status, such as whether a person is a smoker, non-smoker, or prior smoker; medical orders; orders for laboratory test and/or laboratory test results; current and/or past prescription medications; current and/or past treatments; medical history information; insurance coverage, account, and/or billing information; and/or admission and/or discharge information. It will be understood that this list, and all other lists in the Detailed Description, are not exhaustive in nature and therefore, are not to be construed as limiting.

Personally identifying information may be encoded as data in electronic data messages, and the electronic data messages act as input for computer systems executing data workflows. A Health Level Seven (HL7) message is an example of an electronic data message that includes PHI. Health Level Seven (HL7) is an electronic data messaging protocol that enables messaging between applications across systems and that promotes interoperability between systems. Generally, HL7 messages encode electronic data using American Standard Code for Information Interchange (ASCII). An HL7 message comprises segments of related information. Each segment is independent of the other segments in an HL7 message, and segments may be optional or required depending on the type of HL7 message. The order of segments in an HL7 may vary depending on the type of HL7 message, as well. Segments are separated by carriage returns (e.g., <cr>, \r, or \x0D), generally. Each segment is labeled or identified with a header. Exemplary segment headers include MSH (i.e., a message header that conveys the metadata of the HL7 message), PID (i.e., patient identification), NK1 (i.e., next of kin), PV1 (i.e., patient visit), SCH (i.e., scheduling activity information), OBR (i.e., observation request), and/or OBXI (i.e., observation result).

Each segment is divided into fields, and fields are usually separated by one or more vertical bars, known as a pipe character (“I”). The terms “field” and “composite” are used interchangeability herein. A segment may include any number and type of field for information relating to that segment. Each field has a position in the segment. For example, in a PID segments having three fields, the three fields would be identified “PID one,” “PID two,” and “PID three,” wherein one, two, and three refer to a field's placement relative to other fields in the PID segment when read in the code from left to right. A PID segment, for example, may include a name field, a date of birth field, and/or address field, each field storing values that are specific to a particular patient associated with an HL7 message. A field may be repeated within a segment, to provide multiple values for the field. For example, an address field may be repeated within a segment to store data for two different addresses associated with the patient being identified in the PID segment (e.g., a tilde character, “˜”, is placed between two different values to indicate that a field is being repeated). Each field contains values to encode information as data for the segment. Information is encoded as data using, generally, alphanumerical values in each field.

Fields may comprise components. The terms “component” and “sub-composite” are used interchangeably herein. For example, a name field may include a first name component (e.g., component values being “JOHN”) and a surname component (e.g., component values being “DOE”). In such an example, the field values would be “JOHN DOE”. Any number of components may be included as related to the field. Field components may comprise subcomponents. The terms “subcomponent” and “sub-sub-composite” are used interchangeably herein. For example, a name field may include a surname component and the surname component may include a suffix subcomponent (e.g., subcomponent values being “SR”), and/or a prefix subcomponent (e.g., values “MR”). Components and/or subcomponents may be separated in the encoded data using one or more accent characters (“̂”), for example. Any number of subcomponents storing data related to a field component may be included in the field component. As such, as the number of subcomponents, field components, and fields encoding data in an HL7 message increases, the more information is encoded in the HL7 message.

Notably, a user is able to fully customize the configuration of HL7 messages by selecting which field components should be included or excluded in each segment in an HL7 message, for example. A user can customize HL7 messages by configuring each of the segments, fields in the each segment, field components in each field, and/or field subcomponents in each field component, included within an HL7 message, and which types of HL7 messages to use. A user may customize an HL7 message by including or excluding various available segments, fields in the each segment, field components in each field, and/or field subcomponents in each field component. As such, the HL7 messaging protocol is highly customizable. Exponentially adding to the innumerable levels of customizable configurations are the many types of HL7 messages that are available. For example, there are approximately 76 message types available in version 2.9 of the HL7 protocol and approximately 85 message types available in version 2.3.1 of the HL7 protocol. Further, message types may include different sub-types as well, adding to the magnitudes of user customization levels available. For example, there are 51 subtypes of the ADT message type (i.e., Admission, Discharge and Transfer message type). An example of a version 2 HL7 message is shown below:

MSH|\hat{ }~\&|ADT|001||EATEST_SOURCE|201301300435||ADT\hat{ }A08|00000000004127874|P|2.3
PID|1|987654111|44445367||PATIENT\hat{ }Jessie\hat{ }||19791016|M||7|333 Scrub TEST
AVE\hat{ }\hat{ }Malvern\hat{ }PA\hat{ }19355\hat{ }USA|GL|(303)555-4499\hat{ }610-555-1111|610-555-
1111|ENG|M|NP|11158493|128788787|9-87654\hat{ }NC||003|CO
PD1|0001|||12345\hat{ }Owen2\hat{ }Test||||||||
NK1|1|Stwinklestar\hat{ }Annie|EMC|333 Scrub TEST AVE\hat{ }\hat{ }Malvern\hat{ }PA\hat{ }19355\hat{ }USA|\hat{ }\hat{ }\hat{ }\hat{ }\hat{ }\hat{ }(303)555-
4499|\hat{ }\hat{ }\hat{ }\hat{ }\hat{ }\hat{ }610-555-1111|C
NK1|2|Stwinklestar\hat{ }Annie|SEL|333 Scrub TEST AVE\hat{ }\hat{ }Malvern\hat{ }PA\hat{ }19355\hat{ }USA|\hat{ }\hat{ }\hat{ }\hat{ }\hat{ }\hat{ }(303)555-
4499|\hat{ }\hat{ }\hat{ }\hat{ }\hat{ }\hat{ }610-555-1111|E|||ASST TEST ENGG|TEST|ZXX|Cerner||||||||||||||||||\hat{ }\hat{ }\hat{ }\hat{ }\hat{ }\hat{ }610-555-1111|123
OMNICELL DRIVE\hat{ }\hat{ }New York\hat{ }NY\hat{ }10021|||||

As should become apparent from reading this Detailed Discussion, HL7 messages encode large amounts of PHI as electronic data. Because PHI is subject to stringent regulations (i.e., Health Insurance Portability and Accountability Act or HIPAA), there is a growing need to ensure the security of personally identifying information when encoded as electronic data in HL7 messages. The present invention provides a new technological function that ensures the security of personally identifying information when encoded as electronic data in HL7 messages and that is not present in prior systems.

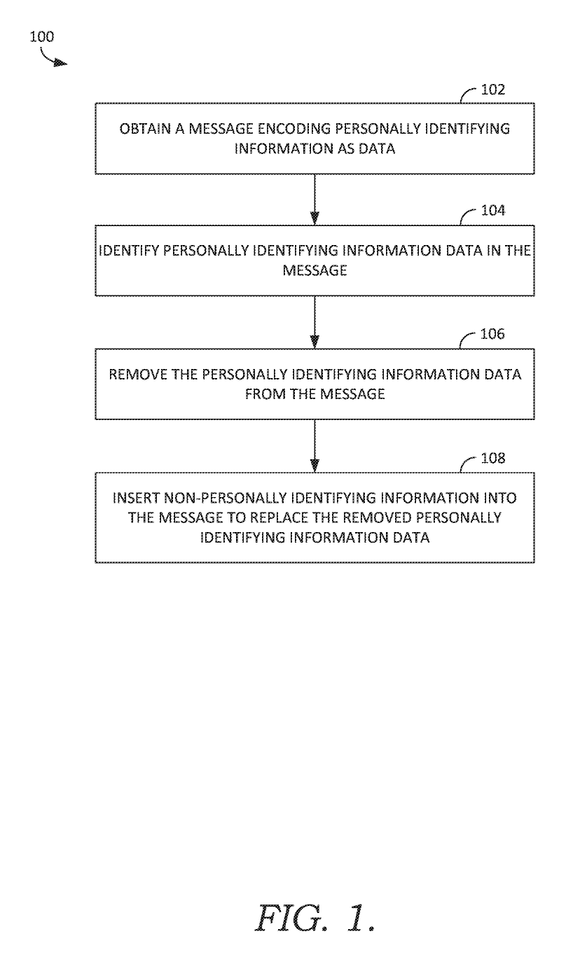

Turning to FIG. 1, a flow diagram of an exemplary method 100 is presented in accordance with an embodiment of the present invention. The method 100 comprises obtaining a message encoding personally identifying information as data, shown at block 102. In some embodiments, the message is a Health Level Seven (HL7) protocol message. The message may be received from an external system or retrieved from a data store, in embodiments. In further embodiments, the method 100 comprises obtaining a plurality of messages, for example, as a set of messages or sets of messages.

At block 104, the method 100 identifies personally identifying information data in the message. Any and/or all personally identifying information encoded in the data may be identified in the message. In some embodiments, personally identifying information data may be distinguished from non-personally identifying information data based, at least in part, on the HL7 messaging protocol. The method 100 may scan or read the data encoded in each HL7 message to locate one or more segments, fields, components, and/or subcomponents in each HL7 message. In this way, the method 100 may identify, within an HL7 message, one or more segments and one or more fields within each of the one or more segments. By locating one or more segments and/or one or more fields in the message, the method 100 may recognize that certain segments and certain fields may contain personally identifying information. For example, the method 100 may recognize a segment in a message having the segment header MSH does not contain personally identifying information whereas a segment having the segment header PID does contain personally identifying information. The method 100 may recognize that a segment having a segment header MSH encodes metadata and is therefore unlikely to contain personally identifying information. In such an embodiment, the method 100 may ‘skip over’ or not scan any data in the segment having the segment header MSH. Additionally or alternatively, method 100 may recognize that a segment having a segment header PID encodes patient information and is therefore very likely to contain personally identifying information. In such an embodiment, the method 100 scans the data in the segment having the segment header PID. It should again be noted that HL7 messages are not standardized, as the HL7 messaging protocol allows for complete customization of message configurations. The configuration of an HL7 message is customizable because every available segment, field in each segment, field component in each field, and/or field subcomponents in field components, can be included or excluded from the message, can be expressed in different value formats, and the sequence of segments, fields, field components, and field subcomponents may be reordered based on message type, for example. In embodiments, the method 100 identifies personally identifying information data in a message at various levels by analyzing one or more segments, one or more fields within each segment, field components and/or field subcomponents. In further embodiments, the method 100 analyzes the data values that encode and represent the personally identifying information data in one or more fields. For example, the method 100 may analyze segment “PID,” locate PHI data in one particular field configured to store name data, and further identify the values “JOHN” are in said field. By analyzing fields, field components, and field subcomponents, the method 100 may recognize a value format is associated with a particular field and/or is associated with specific types of personally identifying information data.

Continuing, at block 106, the method 100 removes the personally identifying information data from the message. In removing the personally identifying information, the method 100 removes one or more values in a corresponding field, field component, and/or field subcomponent wherein the one or more values encode personally identifying information in the message. In removing the personally identifying information data (i.e., values) from the message, the method 100 maintains the value format of the corresponding field, field component, and/or field subcomponent. Accordingly, the method 100 removes values encoding the personally identifying information data in a field, for example, but the field persists in the message for the entry of new values that conform to the value format of the field. The values may be removed by erasing or deleting the values such that the values encoding personally identifying information cannot be recovered.

In embodiments, all of the personally identifying information data in a message is removed. Alternatively, only a portion of personally identifying information data is removed based on a threshold of personally identifying information data, the threshold defining a permissible amount of personally identifying information data, type(s) of personally identifying information data, or a combination thereof to remain in a message. An exemplary threshold might permit personally identifying information data values encoding a zip code or telephone area code to remain in the HL7 message, but would require types of personally identifying information data values such as first name data be removed. In another example, values encoding “ST” or “RD” or “BLVD” in a field subcomponent for patient address information may be permissible (e.g., not removed) PHI. Such ‘innocuous’ PHI may be permissible and not removed, especially, for example, when other field components and/or subcomponents encoding other PHI in the same field, or another related field in the same or different segment, are removed.

Continuing, at block 108, non-personally identifying information is inserted into the message to replace the removed personally identifying information data, in accordance with the method 100. In embodiments, inserting non-personally identifying information into the message to replace the removed personally identifying information data comprises, for each of the one or more fields from which personally identifying information data is removed, generating new values using the value format of the personally identifying information data removed, the new values excluding personally identifying information. Inserting non-personally identifying information into the message to replace the removed personally identifying information data may further comprise, for each of the one or more fields from which personally identifying information data is removed, inserting the non-personally identifying information values into the field.

Although the method 100 discussed herein removes personally identifying information data and inserts non-personally identifying information data, the invention herein contemplates that the steps of removing personally identifying information data and inserting non-personally identifying information data may refer to an overwrite function, wherein the existing values encoding personally identifying information data may be overwritten with values encoding non-personally identifying information data in the message. As such, the invention herein may perform the removal and insertion steps concurrently or simultaneously, such that an overwrite function is contemplated and considered to be within the scope of the invention.

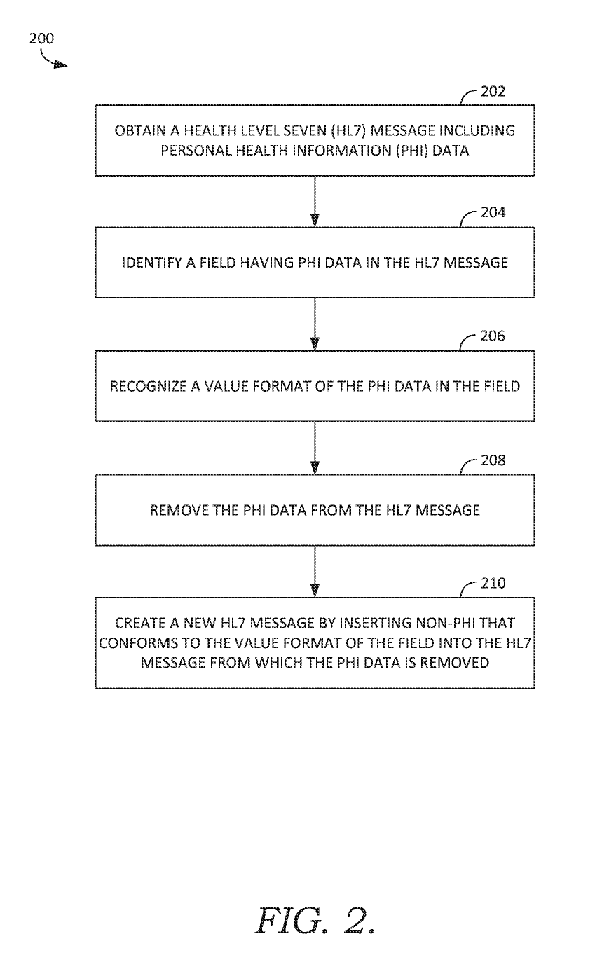

FIG. 2 presents a flow diagram of another exemplary method 200 in accordance with an embodiment of the present invention. In embodiments, one or more non-transitory computer-readable media having computer-executable instructions embodied thereon that, when executed, perform a method as shown in FIG. 2. The method 200 comprises obtaining a Health Level Seven (HL7) message including Personal Health Information (PHI) data, as shown at block 202. The HL7 message(s) may be obtained as previously described with regard to exemplary FIG. 1. In embodiments, the method 200 identifies a field having PHI data in the HL7 message, at block 204. Further, the method 200 may identify all fields within each segment in the HL7 message that include PHI data.

At block 206, the method 200 recognizes a value format of the PHI data in the field. The method 200 may further recognize a value format of a field component and/or field subcomponent. In an embodiment, the method 200 recognizes a value format for all fields within each segment in the HL7 message that include PHI data. In embodiments, recognizing the value format may include identifying a number of characters in the field, identifying a position of the characters relative to one another in the field, identifying when one or more of the characters are grouped together, and/or identifying a relationship between characters when one or more of the characters are grouped together field. For example, a field, field component, and/or field subcomponent may exhibit a value format having six alphanumeric characters (e.g., identifying a number of characters in the field). In another example, a value format may include a grouping and/or position of the characters relative to one another in the field (e.g., dates might be expressed as 20171206 or 12/06/2017 or 06-12-2017), such that four consecutive values encoding a year subcomponent are grouped together and positioned before two consecutive values encoding a month subcomponent, within a date field in an HL7 message. In this way, the method 100 recognizes a value format of the PHI data in a field, field component, and/or subcomponent in an HL7 message.

Continuing, at block 208, the method 200 removes the PHI data from the HL7 message. The PHI data may be removed as discussed above regarding exemplary FIG. 1. At block 210, a new HL7 message is created by inserting non-PHI that conforms to the value format of the field into the HL7 message from which the PHI data is removed. In embodiments, the method 200 generates non-PHI data that conforms with a value format of the field, field component(s), and/or field subcomponent(s). For brevity, value formats are discussed herein with regard to fields; however it will be understood that a value format of a field also refers to and includes value formats of field components, field subcomponents, a sequence of field components, a sequence of field subcomponents, and/or a sequence of field components and subcomponents. For example, the method 200 may generate non-PHI data to conform to the number of characters identified in the field, the position of the characters relative to one another in the field, and/or the relationship between characters identified when one or more of the characters are grouped together. Once non-PHI that conforms to the value format of the field is generated, the non-PHI data values are inserted into respective fields of the HL7 message. In this way, non-PHI data is substituted for and/or used to replace PHI data previously encoded in an HL7 message. In this way, the method 200 creates a new HL7 message. Generally, all of the non-PHI data is concurrently inserted (e.g., at once as opposed to a line-by-line insertion) into all of the fields from which PHI data is removed to create the new HL7 message, in embodiments. At block 210, the method 100 provides the new HL7 message as input to a dataflow. The new HL7 message does not include PHI, or alternatively, includes a permissible type and/or amount of PHI based on a threshold, as previously discussed. In embodiments, the non-PHI data inserted into the new HL7 message is compatible with HL7 messaging protocol.

Additionally or alternatively, the method 200 may locate, in a data store, non-PHI data that corresponds to the field and conforms to the value format of said field. In some embodiments, the method 200 generates a portion of the non-PHI data to be inserted into the message while obtaining another portion of non-PHI from a data store to be inserted into the message. The method 100 may generate non-PHI data for specific fields or segments, in some embodiments. The method 100 may retrieve pre-formatted or previously generated non-PHI data for specific fields or segments, in some embodiments.

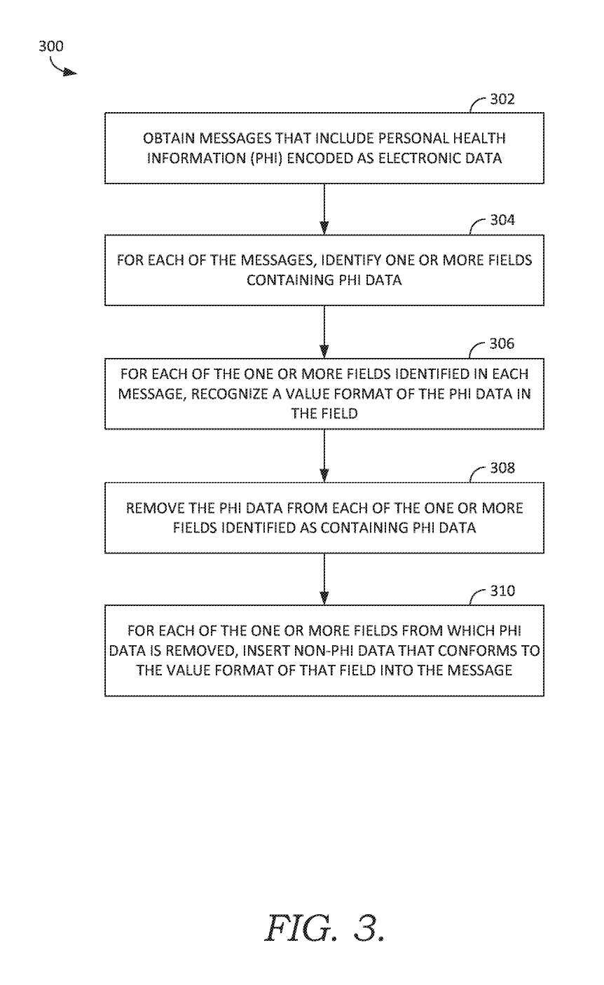

FIG. 3 is a flow diagram of an exemplary method 300 in accordance with an embodiment of the present invention. In embodiments, the method 300 is performed using one or more non-transitory computer-readable media having computer-executable instructions embodied thereon that, when executed, perform the method 300. The method 300 comprises obtaining messages that include Personal Health Information (PHI) encoded as electronic data, shown at block 302. For each of the messages, the method 300 comprises identifying one or more fields containing PHI data, illustrated at block 304. In embodiments, PHI data in the HL7 message may be recognized as distinguishable from non-PHI data in that same HL7 message based on user-defined configurations. Additionally or alternatively, the PHI data in an HL7 message may be recognized, as opposed to non-PHI data in the message, based on the technological techniques discussed hereinabove with regard to exemplary FIGS. 1 and 2.

For each of the one or more fields identified as containing PHI data in each message, the method 300 recognizes a value format of the PHI data in the field, at block 306. At block 308, the method 300 removes the PHI data from each of the one or more fields identified as containing PHI data. In some embodiments, the method 300 retrieves, from a data store, non-PHI data that corresponds to the field and conforms to the value format of the field from which PHI data is removed, for example. For each of the one or more fields from which PHI data is removed, the method 300 comprises inserting non-PHI data that conforms to the value format of that field into the message, shown at block 310. When inserting non-PHI data, the method 300 may simultaneously or concurrently insert all of the non-PHI data into all of the fields from which PHI was removed from an HL7 message. In some embodiments, the method 300 provides the new HL7 message as input to a dataflow.

In further embodiments, the method 300 obtains multiple messages. The method 300 may recognize when two or more of the messages are related by PHI. For example, two different HL7 messages may include the same medical record number, the same patient name, the same phone number, and same address, or the like. The method 300 may exploit this relation in order to remove PHI data and insert non-PHI data into the two or messages. For example, the same PHI data values may be inserted into corresponding fields of the two different HL7 messages. This reduces the need to generate, retrieve, or otherwise produce unique instances of non-PHI data. As such, in an embodiment, the method 300 analyzes the messages including PHI and recognizes when two or messages share the same or similar PHI. The method 300 may associate the two or more of the messages that contain the same PHI data. Then, for each of the one or more fields from which PHI data is removed from the two or more associated messages, the method 300 inserts the same non-PHI data (e.g., identical non-PHI data values) into the two or more associated messages. Using this association, the method 300 may build sets of HL7 messages that correspond to one test patient, for example. Sets of HL7 messages that corresponds to one test patient may be advantageously used as input for testing a computerized workflow, for example. Alternatively, when two or more of the messages contain the same PHI data, the method 300 may insert non-identical non-PHI data into the two or more messages for each of the one or more fields from which PHI data is removed for the two or more messages. In this way, diverse test patient data/multiple test patients may be generated, thus solving the technological problem of data scarcity (e.g., insufficient patient data available as input for testing a computerized workflow).

It will be appreciated by those having ordinary skill in the art that the exemplary embodiments discussed above with regard to each FIGS. 1, 2, and 3 may be implemented using hardware, software, memory, processor(s), and/or computer device(s) in a centralized and/or distributed computing environment, as referenced hereinafter with regard to FIG. 21. The embodiments of the present invention may further be implemented through a web browser or an application programming interface (API), for example, accessible by users through graphical user interfaces (GUI), as discussed hereinafter.

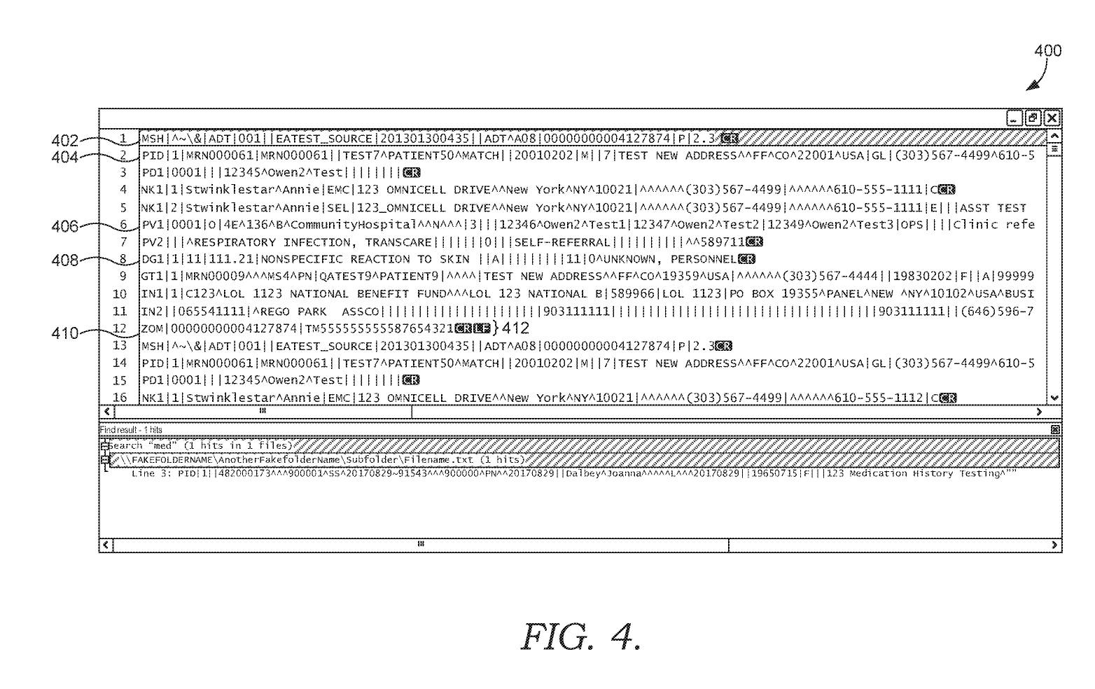

FIG. 4 depicts an exemplary GUI 400 in accordance with embodiments of the present invention. As shown in FIG. 4, an exemplary HL7 message encodes personally identifying information, such as PHI. The HL7 message includes several segments, identified with segment headers such as MSH 402, PID 404, PVI 406, DG1 408, and ZOM 410, for example. Messages that are utilized in the claimed embodiments discussed herein may also include a special, non-standardized segment 412 (e.g., ZOM 410), that encodes a unique identifier for the message. This non-standardized segment 412 is used, in some embodiments of the present invention, to track and store the HL7 message in a database once PHI data has been replaced with non-PHI data, for example. The exemplary HL7 message shown in FIG. 4 includes a non-standardized segment 412. Each segment includes one or more fields, one or more field components, and/or one or more subcomponents. Regarding encoding data using a computer readable language, fields are separated with pipe character(s) and field components/subcomponents are separated with accent character(s) as previously discussed hereinabove.

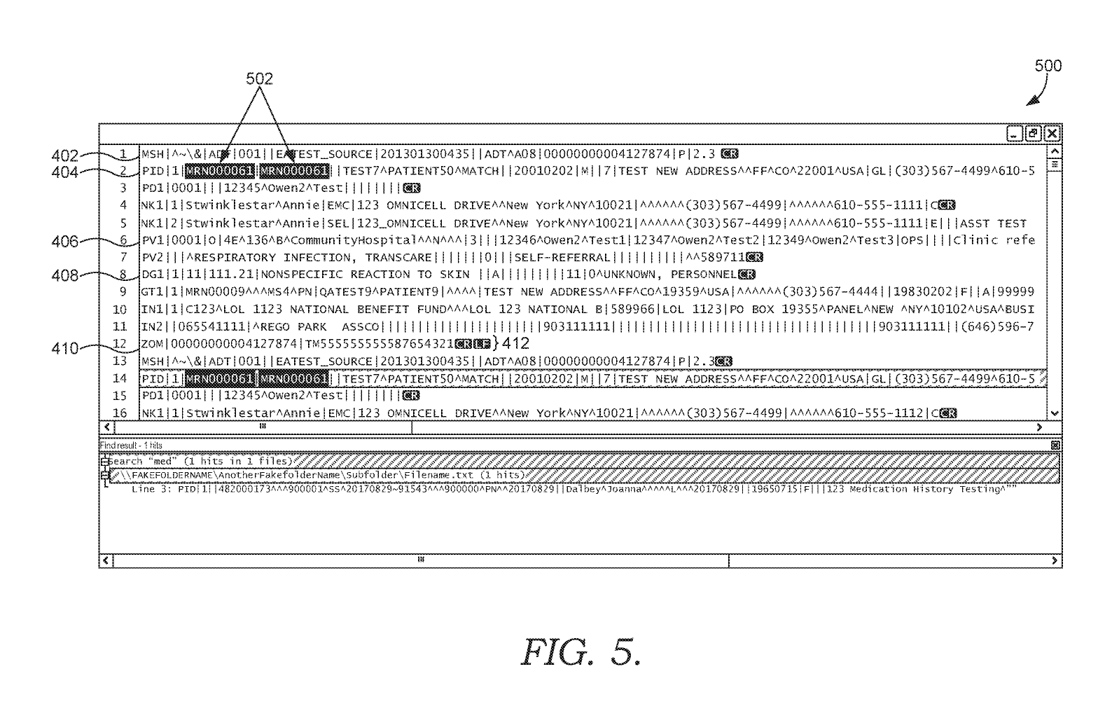

Continuing, FIG. 5 depicts an exemplary GUI 500 in accordance with embodiments of the present invention. Using embodiments of the present invention, one or more fields may be visually highlighted on the GUI 500, for example, to indicate a particular type of PHI. In example of FIG. 5, the PHI of a medical record number, MRN 502, is highlighted in the PID segment. In embodiments, different fields of PHI may be highlighted on the GUI 500 in this manner to visually draw attention to PHI fields repeated through one or more HL7 messages, for example.

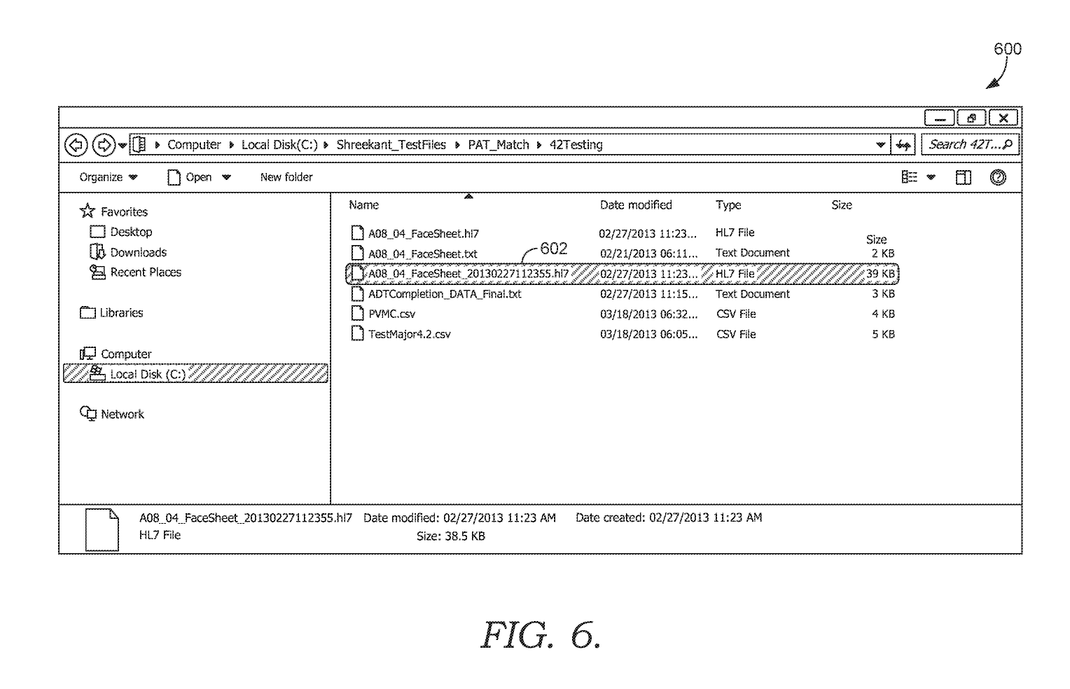

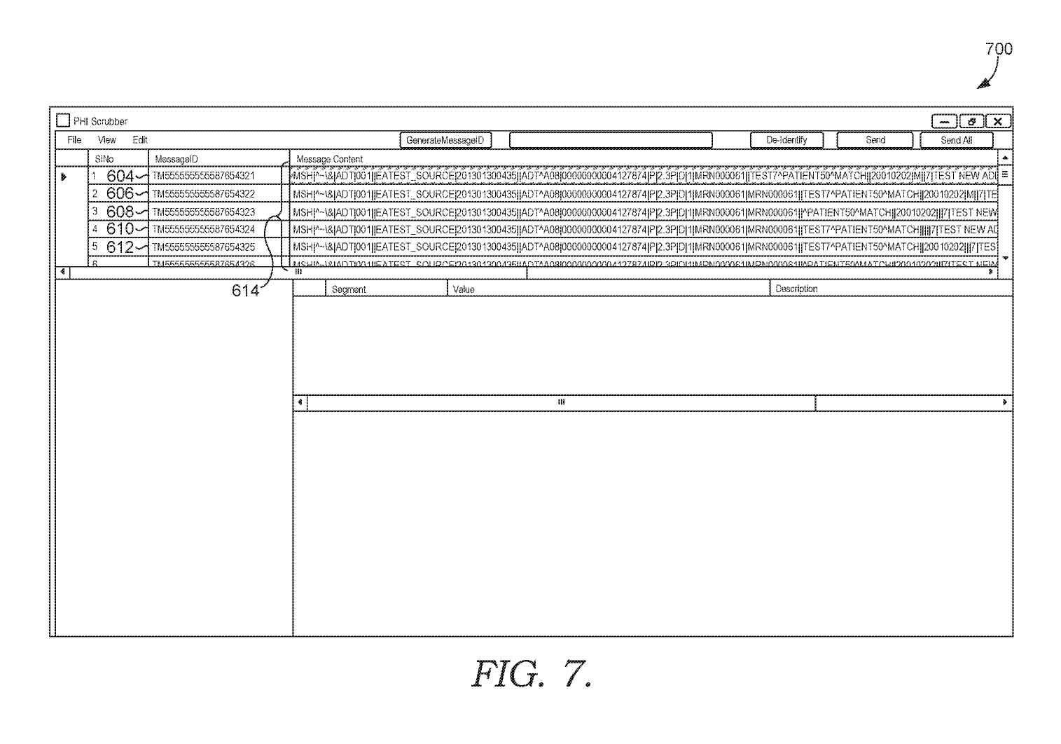

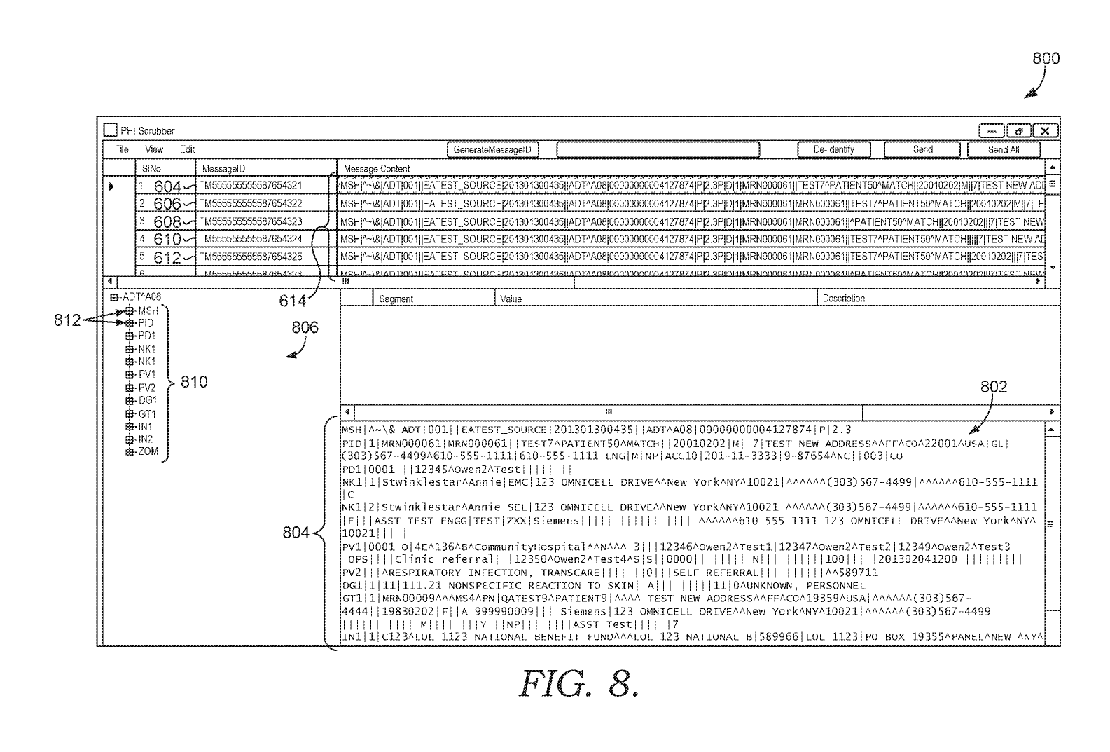

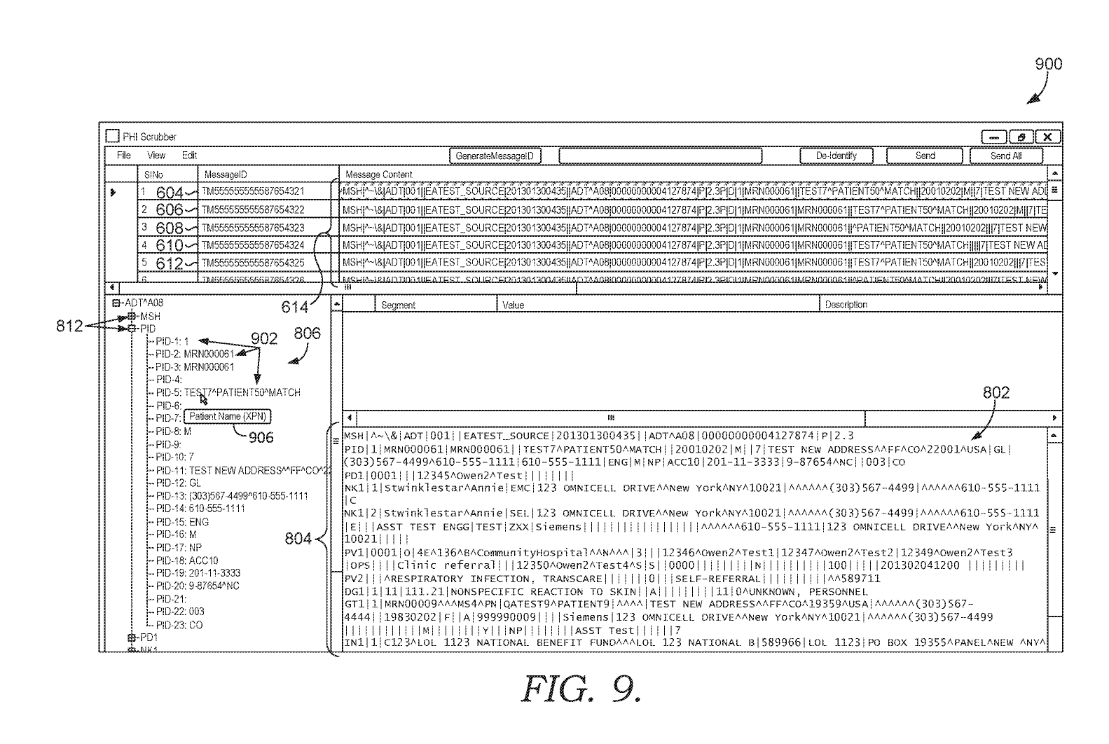

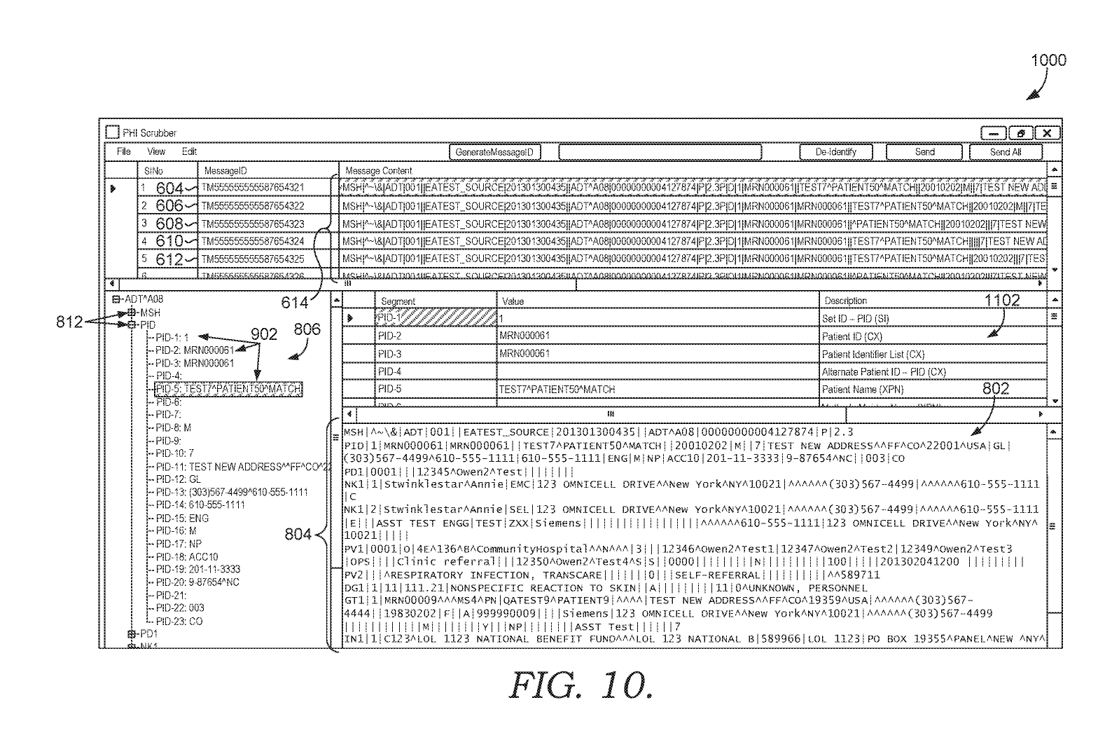

Having illustrated an example of an HL7 message, an exemplary GUI 600 is shown in FIG. 6, in accordance with embodiments of the present invention. In FIG. 6, a user may open a particular HL7 file 602 containing one or more HL7 messages, by interacting with the GUI 600. In this way, HL7 messages may be obtained, for example. The HL7 file 602 is opened, as shown in FIG. 7, which depicts an exemplary GUI 700 in accordance with embodiments of the present invention. As opened, the HL7 file 602 comprises a plurality of HL7 messages, each of the plurality of HL7 messages having an identifier 604, 606, 608, 610, and 612, for example. Each of the HL7 messages may contain PHI corresponding to the same patient, or different patients, in various embodiments. At least a portion of the data encoded in the plurality of messages may be presented, such that the GUI 700 provides a preview area 614 for one or more of the plurality of HL7 messages 604, 606, 608, 610, and 612, for example. At FIG. 8, an exemplary GUI 800 is illustrated in accordance with embodiments of the present invention. One of the HL7 messages 606 is presented in a detailed view area 802, such that the encoded data 804 is visible and may be scrolled through by a user. The detailed view area 802 may be populated based on a selection of a message in the preview area 614, for example. The detailed view area 802 is presented separately from the preview area 614. Additionally, a message node area 806 is presented separately from the detailed view area 802 and the preview area 614. The message node area 810 presents nodes 812 for each segment contained in the HL7 message 606 using the segment headers of each segment. The nodes 812 may be selectable. As shown in FIG. 9, which depicted another exemplary GUI 900 in accordance with embodiments of the present invention, selection of one of the nodes 812, in this example, segment PID, triggers the presentation in the GUI 900 of subnodes for fields, field components, and/or field subcomponents 902 of the node (e.g., PID fields labeled numerically as 1, 5, and 7) that are encoded in that particular segment. In FIG. 9, a user hovers a cursor 904 over subnode PID-5, causing a popup 906 to be displayed. The popup 906 may present information corresponding to the subnode, in embodiments, such as a description of data encoded in a field that corresponds to that particular subnode. When a user engages or selects a subnode, as shown in the exemplary GUI 1000 presented in FIG. 10, a selected subnode (i.e., PID-5) is highlighted to indicate its selection and a segment detail area 1002 is populated with information from the corresponding segment of the message.

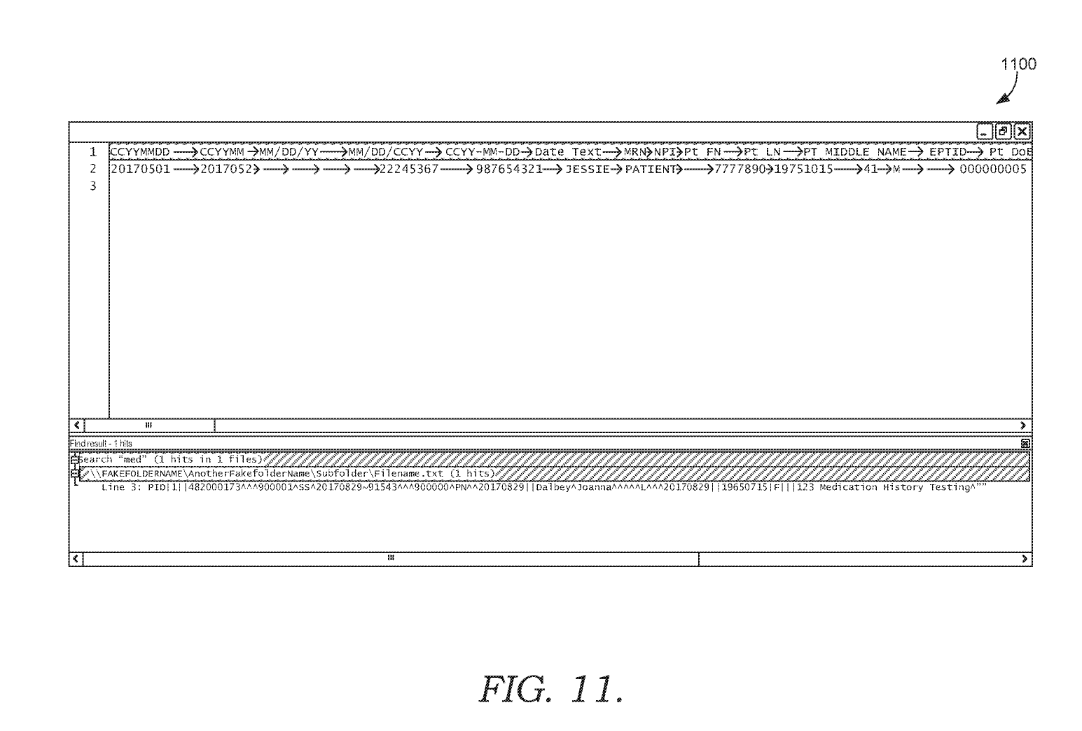

In embodiments of the present invention, the value formats of each field, field component, and/or field subcomponent are recognized by analyzing the HL7 message 606, as previously discussed. For example, FIG. 11 depicts an exemplary GUI 1100 in accordance with embodiments of the present invention. In FIG. 11, available value formats of different fields in a segment are presented in line 1 as recognized by analyzing line 2, which presents data encoding PHI. For example, a field designated as “MRN” in line 1 corresponds to the data values “22245367” encoding PHI in line 2 and another field or field component designated as “Pt FN” (e.g., patient first name) in line corresponds to the data values “JESSIE” encoding PHI in line 2.

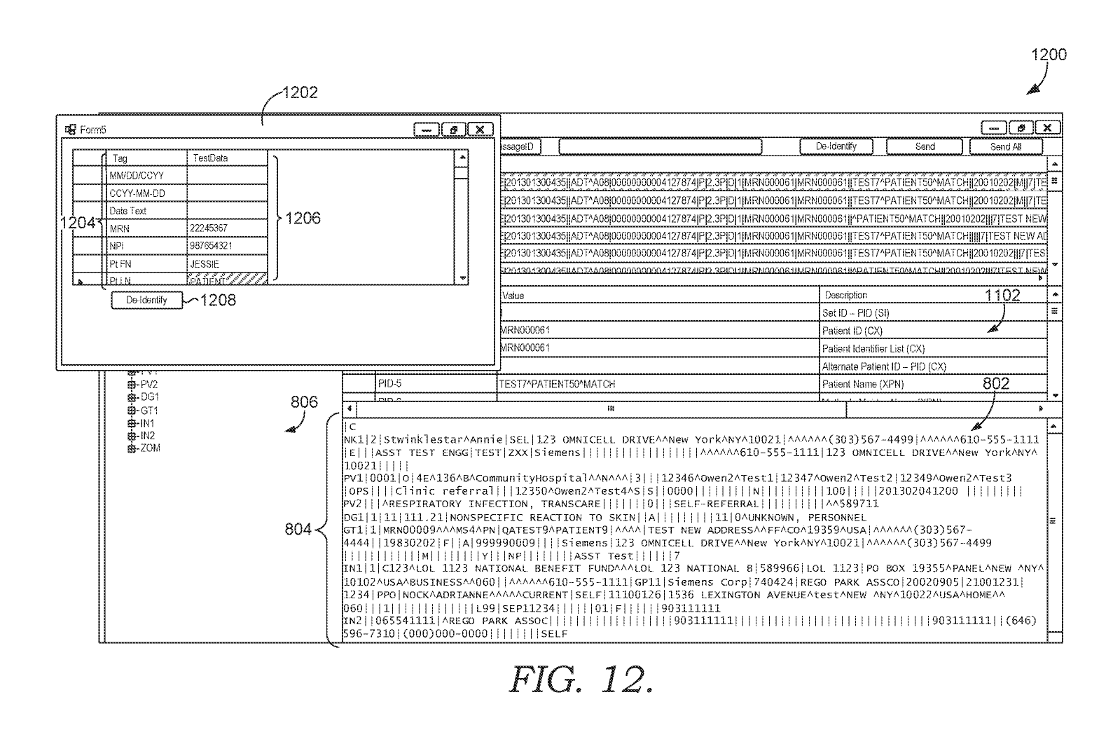

Turning to FIG. 12, it depicts an exemplary GUI 1200 in accordance with the embodiments of the present invention. At FIG. 12, embodiments of the present invention are analyzing the HL7 message 606, for example, and identifying segments, fields, field components, and/or field subcomponents. Based on the analysis, PHI encoded in the data of said HL7 message 606 is identified. The analysis may also present an analysis area 1202 to present identified fields, field components, and/or field subcomponents 1204 (e.g., Pt FN) and the data values 1206 (e.g., JESSIE) encoded for those fields, field components, and/or field subcomponents. In one example, a field designated as “MRN” corresponds to the data values “22245367” encoding PHI, as previously indicated in lines 1 and 2 of exemplary FIG. 11. A user may scroll through the various analysis results, as shown in FIG. 12. In various instances, some fields, field components, and/or field subcomponents are not present in a message as there is no corresponding data encoded in the message for those fields, field components, and/or field subcomponents. The analysis area 1202 includes a user interface object 1208 that is selectable. Selection or engagement of the user interface object 1208 initiates the removal of PHI from the message.

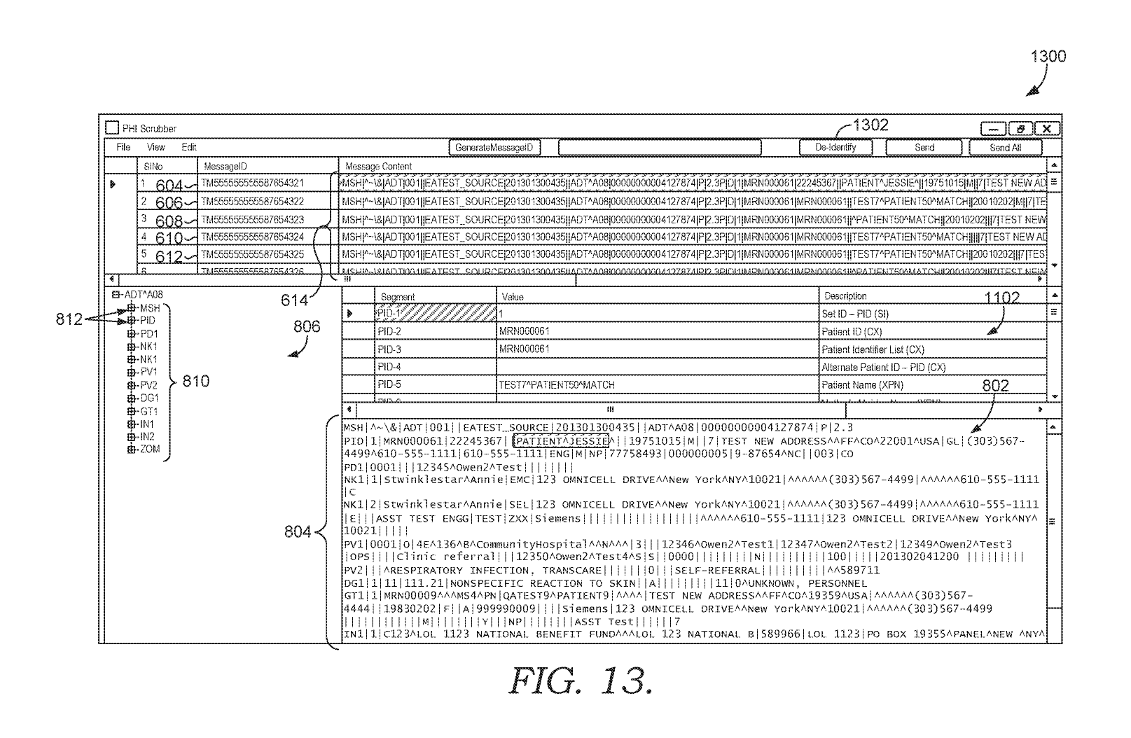

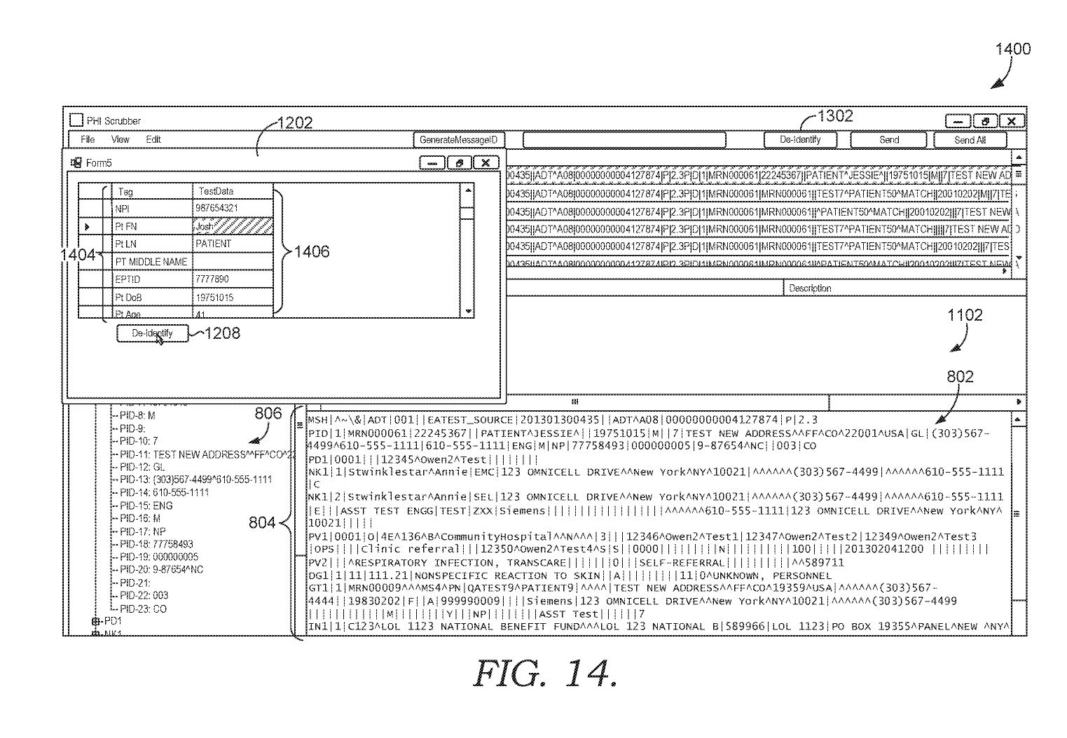

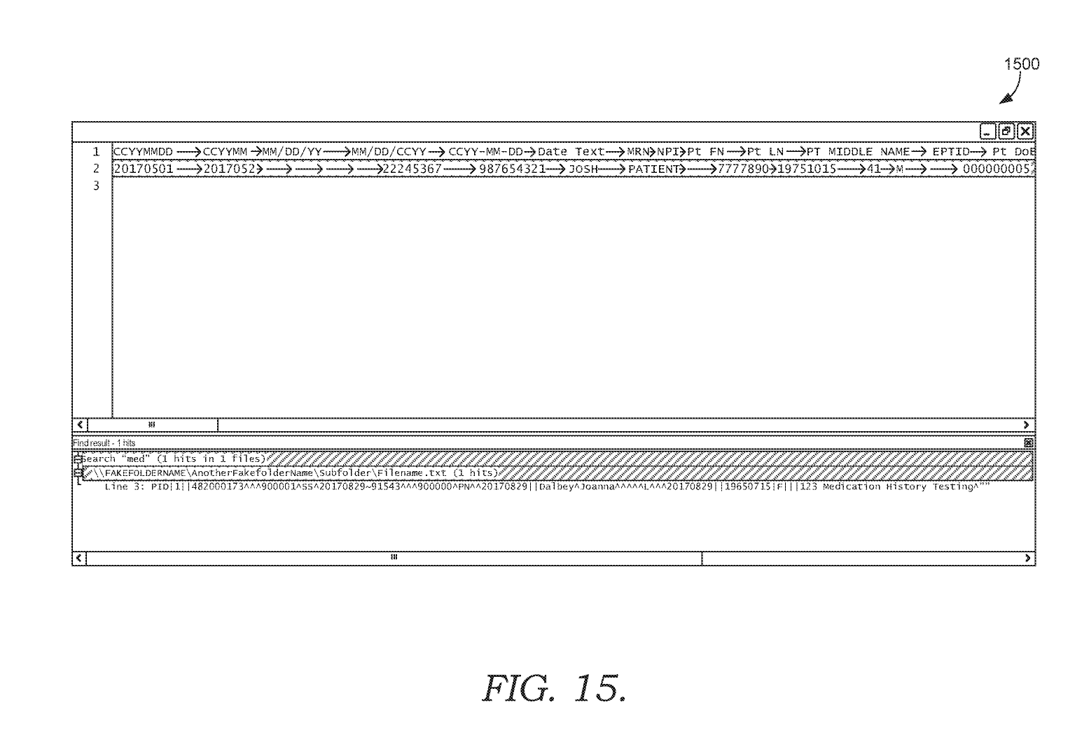

FIG. 13 depicts an exemplary GUI 1300 in accordance with embodiments of the present invention. As shown in FIG. 13, the HL7 message 606 includes PHI for a patient, the patient's name being encoded with alphanumeric values, as JESSIE PATIENT in the PID segment, among other PHI information. In order to remove the PHI, a user may engage or select a user interface object 1302. Turning to FIG. 14, which depicts an exemplary GUI 1400 in accordance with embodiments of the present invention, in response to engagement of user interface object 1302 to remove the PHI from the message, non-PHI data is generated or retrieved, and presented in the analysis area 1202 for review, for example. As shown in the analysis area 1202, the “dummy data” or non-PHI data is presented for fields, field components, and/or field subcomponents 1404 (e.g., Pt FN) and the data values 1206 (e.g., JOSH) to be encoded for those fields, field components, and/or field subcomponents. Notably, the non-PHI data is presented for fields, field components, and/or field subcomponents 1404 (e.g., Pt FN) and the data values 1206 (e.g., JOSH) to be encoded for those fields, field components, and/or field subcomponents follow the value formats originally identified for the HL7 message 606, as shown in the analysis area 1202 in FIG. 12. For example, as shown in the exemplary GUI 1500 of FIG. 15, the information presented in line 1 includes the same fields and the same values formats shown in exemplary FIG. 11, with regard to HL7 message 606. However, in FIG. 15, line 2 presents the non-PHI data that is used to replace the PHI data removed from the HL7 message 606.

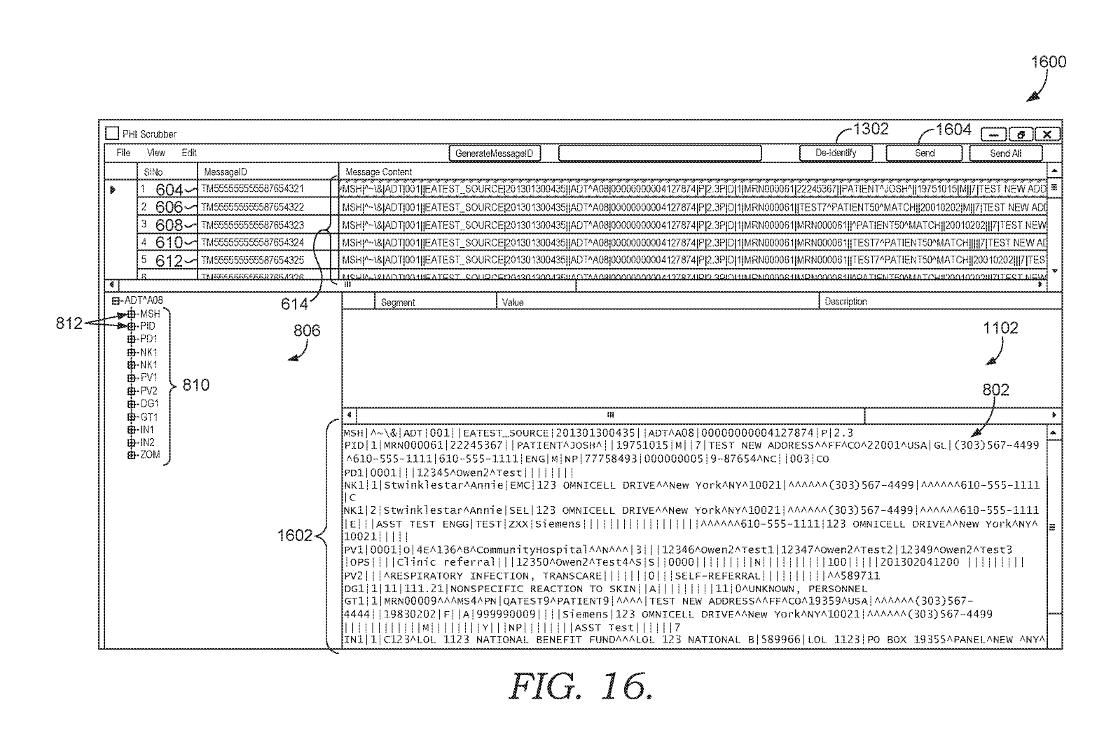

Turning back to GUI 1400, when the user interface object 1208 in the analysis area 1202 is engaged or selected, the PHI data is removed from the segments, fields, field components, and/or field subcomponents of the HL7 message 606. The non-PHI data is inserted into the HL7 message, thus creating a new HL7 message encoding non-PHI data that conforms to the original value formats of the original PHI that has been removed. At FIG. 16, the exemplary GUI 1600 presents the new HL7 message 1602, shown in the detailed view area 802. The new HL7 message 1602 has been populated with the non-PHI data (e.g., data values JESSIE are changed to JOSH).

Once PHI data is removed and non-PHI data is inserted to create a new HL7 message, the new HL7 message may be provided as input to a data flow. Because personally identifying information encoded as PHI data in the HL7 message has been removed, security of the personally identifying information is not compromised. Additionally, the new HL7 message may be provided as input to the workflow because it conforms with the appropriate value formats of the original PHI data. For example, a user may engage a graphical user interface object, such as the exemplary send button 1604, in order to communicate the new HL7 message to another computer system.

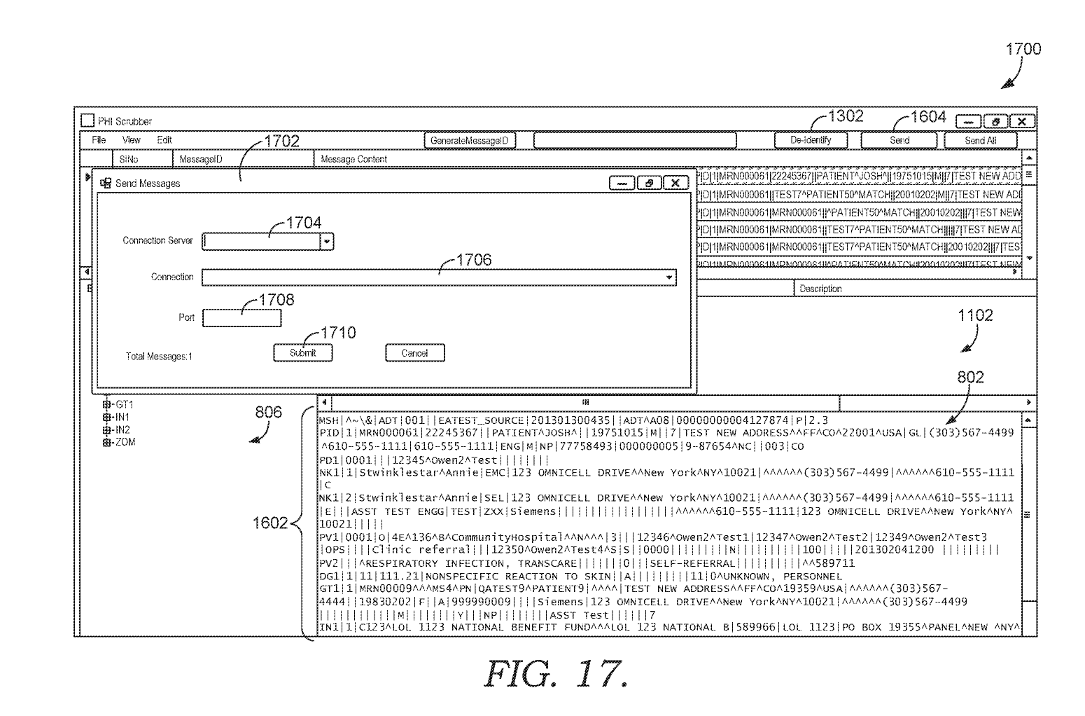

Having engaged the send button 1604, for example, a communication popup window 1702 is displayed to a user as shown in the exemplary GUI 1700 of FIG. 17, in accordance with embodiments of the present invention. The new HL7 message shown in the detail view area 802 may be sent to another entity to serve as input to a dataflow that uses HL7 messaging protocol. The communication popup window 1702 includes input fields 1704, 1706, and 1708. A user many input information or a drop down menu with user selectable options may be displayed under each of the input fields 1704, 1706, and 1708. In this way, a user may specify a particular server, connection, and/or port through which the new HL7 message may be sent to another computer system, as input. The user may select a submit button 1710, for example, to communicate the new HL7 message to another computer system.

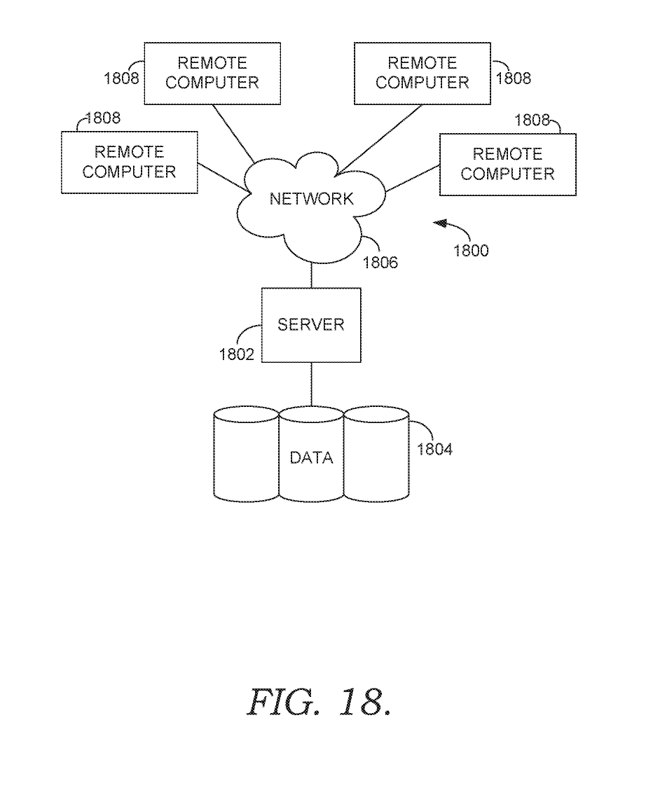

Finally, continuing to FIG. 18, it depicts a block diagram of an exemplary computing environment 1800 suitable to implement embodiments of the present invention. It will be understood by those of ordinary skill in the art that the exemplary computing environment 1800 is just one example of a suitable computing environment and is not intended to limit the scope of use or functionality of the present invention. Similarly, the computing environment 1800 should not be interpreted as imputing any dependency and/or any requirements with regard to each component and combination(s) of components illustrated in FIG. 18. It will be appreciated by those having ordinary skill in the art that the connections illustrated in FIG. 18 are also exemplary as other methods, hardware, software, and devices for establishing a communications link between the components, devices, systems, and entities, as shown in FIG. 18, may be utilized in implementation of the present invention. Although the connections are depicted using one or more solid lines, it will be understood by those having ordinary skill in the art that the exemplary connections of FIG. 18 may be hardwired or wireless, and may use intermediary components that have been omitted or not included in FIG. 18 for simplicity's sake. As such, the absence of components from FIG. 18 should be not be interpreted as limiting the present invention to exclude additional components and combination(s) of components. Moreover, though devices and components are represented in FIG. 18 as singular devices and components, it will be appreciated that some embodiments may include a plurality of the devices and components such that FIG. 18 should not be considered as limiting the number of a device or component.

Continuing, the computing environment 1800 of FIG. 18 is illustrated as being a distributed environment where components and devices may be remote from one another and may perform separate tasks. The components and devices may communicate with one another and may be linked to each other using a network 1802. The network 1802 may include wireless and/or physical (e.g., hardwired) connections. Exemplary networks include a telecommunications network of a service provider or carrier, Wide Area Network (WAN), a Local Area Network (LAN), a Wireless Local Area Network (WLAN), a cellular telecommunications network, a Wi-Fi network, a short range wireless network, a Wireless Metropolitan Area Network (WMAN), a Bluetooth® capable network, a fiber optic network, or a combination thereof. The network 1802, generally, provides the components and devices access to the Internet and web-based applications.

The computing environment 1800 comprises a computing device in the form of a server 1804. Although illustrated as one component in FIG. 18, the present invention may utilize a plurality of local servers and/or remote servers in the computing environment 1800. The server 1804 may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server 1804 may include or may have access to computer-readable media. Computer-readable media can be any available media that may be accessed by server 1804, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 1804. Computer storage media does not comprise signals per se.

Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, radio frequency (RF), infrared, and other wireless media. Combinations of any of the above also may be included within the scope of computer-readable media.

In embodiments, the server 1804 uses logical connections to communicate with one or more remote computers 1806 within the computing environment 1800. In embodiments where the network 1802 includes a wireless network, the server 1804 may employ a modem to establish communications with the Internet, the server 1804 may connect to the Internet using Wi-Fi or wireless access points, or the server may use a wireless network adapter to access the Internet. The server 1804 engages in two-way communication with any or all of the components and devices illustrated in FIG. 18, using the network 1802. Accordingly, the server 1804 may send data to and receive data from the remote computers 1806 over the network 1802.

Although illustrated as a single device, the remote computers 1806 may include multiple computing devices. In an embodiment having a distributed network, the remote computers 1806 may be located at one or more different geographic locations. In an embodiment where the remote computers 1806 is a plurality of computing devices, each of the plurality of computing devices may be located across various locations such as buildings in a campus, medical and research facilities at a medical complex, offices or “branches” of a banking/credit entity, or may be mobile devices that are wearable or carried by personnel, or attached to vehicles or trackable items in a warehouse, for example.

In some embodiments, the remote computers 1806 is physically located in a medical setting such as, for example, a laboratory, inpatient room, an outpatient room, a hospital, a medical vehicle, a veterinary environment, an ambulatory setting, a medical billing office, a financial or administrative office, hospital administration setting, an in-home medical care environment, and/or medical professionals' offices. By way of example, a medical professional may include physicians; medical specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; veterinarians; students; and the like. In other embodiments, the remote computers 1806 may be physically located in a non-medical setting, such as a packing and shipping facility or deployed within a fleet of delivery or courier vehicles.

Continuing, the computing environment 1800 includes a data store 1808. Although shown as a single component, the data store 1808 may be implemented using multiple data stores that are communicatively coupled to one another, independent of the geographic or physical location of a memory device. Exemplary data stores may also store data in the form of electronic records, for example, electronic medical records of patients, transaction records, billing records, task and workflow records, chronological event records, and the like.

Generally, the data store 1808 includes physical memory that is configured to store information encoded in data. For example, the data store 1808 may provide storage for computer-readable instructions, computer-executable instructions, data structures, data arrays, computer programs, applications, and other data that supports the functions and action to be undertaken using the computing environment 1800 and components shown in exemplary FIG. 18.

In a computing environment having distributed components that are communicatively coupled via the network 1802, program modules may be located in local and/or remote computer storage media including, for example only, memory storage devices. Embodiments of the present invention may be described in the context of computer-executable instructions, such as program modules, being executed by a computing device. Program modules may include, but are not limited to, routines, programs, objects, components, and data structures that perform particular tasks or implement particular data types. In embodiments, the server 1804 may access, retrieve, communicate, receive, and update information stored in the data store 1808, including program modules. Accordingly, the server 1804 may execute, using a processor, computer instructions stored in the data store 1808 in order to perform embodiments described herein.

Although internal components of the devices in FIG. 18, such as the server 1804, are not illustrated, those of ordinary skill in the art will appreciate that internal components and their interconnection are present in the devices of FIG. 18. Accordingly, additional details concerning the internal construction device are not further disclosed herein.

The present invention has been described in relation to particular embodiments, which are intended in all respects to be illustrative rather than restrictive. Further, the present invention is not limited to these embodiments, but variations and modifications may be made without departing from the scope of the present invention.

What is claimed is:

1. A computerized method comprising:
obtaining a message encoding personally identifying information as data;
identifying personally identifying information data in the message;
removing the personally identifying information data from the message; and
inserting non-personally identifying information into the message to replace the removed personally identifying information data.

2. The computerized method of claim 1 further comprising:
identifying one or more fields in the message; and
identifying personally identifying information data in the one or more fields.

3. The computerized method of claim 2 further comprising:
analyzing values of the personally identifying information data in the one or more fields to identify, for each of the one or more fields, a value format of the personally identifying information data in the field.

4. The computerized method of claim 3, wherein removing the personally identifying information data from the message further comprises:
maintaining the value format of the one or more fields while removing values of the personally identifying information data in the one or more fields.

5. The computerized method of claim 4, wherein inserting non-personally identifying information into the message to replace the removed personally identifying information data further comprises:
for each of the one or more fields from which personally identifying information data was removed, generating new values using the value format of the personally identifying information data removed, the new values excluding personally identifying information; and
for each of the one or more fields from which personally identifying information data was removed, inserting the non-personally identifying information values into the field.

6. The computerized method of claim 1, wherein the message is a Health Level Seven (HL7) protocol message.

7. One or more non-transitory computer-readable media having computer-executable instructions embodied thereon that, when executed, perform a method comprising:
obtaining a Health Level Seven (HL7) message including Personal Health Information (PHI) data;
identifying a field having PHI data in the HL7 message;
recognizing a value format of the PHI data in the field;
removing the PHI data from the HL7 message; and
creating a new HL7 message by inserting non-PHI that conforms to the value format of the field into the HL7 message from which the PHI data is removed.

8. The media of claim 7, wherein recognizing the value format of the PHI data in the field further comprises one or more of:
identifying a number of characters in the field;
identifying a position of the characters relative to one another in the field;
identifying when one or more of the characters are grouped together; and
identifying a relationship between characters when one or more of the characters are grouped together field.

9. The media of claim 8, wherein the method further comprises:
generating non-PHI data that conforms one or more of: the number of characters identified in the field, the position of the characters relative to one another in the field, the relationship between characters identified when one or more of the characters are grouped together.

10. The media of claim 7, wherein the method further comprises:
identifying a field having PHI data in the HL7 message further comprises identifying all fields within each segment in the HL7 message that include PHI data; and
wherein recognizing a value format of the PHI data in the field further comprises recognizing a value format for all fields within each segment in the HL7 message that include PHI data.

11. The media of claim 10, wherein the method further comprises:
locating, in a data store, non-PHI data that corresponds to the field and conforms to the value format of said field.

12. The media of claim 11, wherein non-PHI data is concurrently inserted into all fields from which PHI data removed to create the new HL7 message.

13. The media of claim 7, wherein the method further comprises:
providing the new HL7 message as input to a dataflow.

14. One or more non-transitory computer-readable media having computer-executable instructions embodied thereon that, when executed, perform a method comprising:
obtaining messages that include Personal Health Information (PHI) encoded as electronic data;
for each of the messages, identifying one or more fields containing PHI data;
for each of the one or more fields identified in each message, recognizing a value format of the PHI data in the field;
removing the PHI data from each of the one or more fields identified as containing PHI data; and
for each of the one or more fields from which PHI data is removed, inserting non-PHI data that conforms to the value format of that field into the message.

15. The media of claim 14, wherein the method further comprises:
associating two or more of the messages that contain the same PHI data; and
for each of the one or more fields from which PHI data is removed for the two or more associated messages, inserting identical non-PHI data into the two or more associated messages.

16. The media of claim 15, wherein the method further comprises:
retrieving, from a data store, non-PHI data that corresponds to the field and conforms to the value format of said field; and
when inserting non-PHI data, concurrently inserting non-PHI data into all fields from which PHI was removed from an HL7 message.

17. The media of claim 14, wherein the method further comprises:
wherein PHI in the HL7 message is recognized as distinguishable from non-PHI in the HL7 message based on user-defined configurations.

18. The media of claim 14, wherein the non-PHI data inserted into the new HL7 message is compatible with HL7 messaging protocol.

19. The media of claim 14, wherein the method further comprises:
when two or more of the messages contain the same PHI data, inserting non-identical non-PHI data into the two or more messages for each of the one or more fields from which PHI data is removed for the two or more messages.

20. The media of claim 14, wherein the method further comprises:
providing the new HL7 message as input to a dataflow.
